(19) United States
(12) Patent Application Publication
(10) Pub. No.: US 2020/0345444 A1
(43) Pub. Date: Nov. 5, 2020

(54) ORTHOPEDIC IMPLANT CARRIER SYSTEM AND METHOD

(71) Applicant: Nextremity Solutions, Inc., Warsaw, IN (US)

(72) Inventor: Adam FINLEY, Warsaw, IN (US)

(73) Assignee: Nextremity Solutions, Inc., Warsaw, IN (US)

(21) Appl. No.: 16/865,991

(22) Filed: May 4, 2020

Related U.S. Application Data
(60) Provisional application No. 62/842,830, filed on May 3, 2019.

(51) Int. Cl. A61B 50/31

(52) U.S. Cl. CPC A61B 50/31; A61B 2050/005

(57) ABSTRACT

An orthopedic implant carrier system has a carrier with a front cover, a back cover, and a linking member pivotally connected to the front cover and the back cover, such that the front cover and back cover are opposably movable about the linking member, with respect to each other. At least one insert connector is connected to the carrier, with an insert releasably connectable to the at least one insert connector. The insert has a pocket, with a plurality of orthopedic implants organized by implant type, size, and length.

### CROSS-REFERENCE TO RELATED APPLICATIONS

The present application claims priority to U.S. Provisional Application No. 62/842,830 filed on May 3, 2019, which is incorporated herein by referenced in its entirety.

### FIELD OF THE INVENTION

This application relates generally to apparatuses, devices, and methods for storing, organizing, and transporting, implants and more particularly to apparatuses, devices, and methods for storing, organizing, and transporting orthopedic implants that are sterilized.

### DESCRIPTION OF THE RELATED ART

Potential contamination is a concern in a medical environment, particularly when objects are transported from a non-sterile space to a sterile space. Implants for use in orthopedic surgery are commonly in packages, that include a hard-shell box, blister shells, bags, tubes or combinations, with the implant kept sterile inside. Implants may also be left unpackaged but placed on a tray, where the tray and contents are sterilized before use. Hard-shell boxes and tubes are popular but large numbers of such implant containers require a large carrier or several carriers due to the large volume taken-up by such implant containers. Such individual implants or packages may be transported using a case or box, commonly referred to as a screw caddy or an implant caddy. Implant caddies and similar transporting devices may be quite large and bulky. To transport a full assortment of surgical implants requires slots for upwards of 400 implants. For 400 implants, at least five standard implant caddies would be required. Implants and packaging are generally placed in individual slots of such a caddy, which often makes it difficult for a surgeon to identify implant types, implant size, and implant length. This may cause delays during surgery. Individual screws are also difficult to organize and because of the generally small size, and mistakes may be made selecting an implant.

When surgical implants are in tubes or individual packages, they may also be selected for use prior to entering a sterile environment. With most packaging, there is at least a double layer of packaging, and in some cases even an outer blister shell. This poses a problem because the outer surface of the packaging is non-sterile. Opening a package to retrieve a surgical implant may require multiple people, with one person only handling the non-sterile portions of packaging and second person handling the sterile portions of packaging and taking care not to contact the non-sterile portions. Another difficulty exists if an incorrect set of implants is selected and additional implants need to be brought from storage, through the non-sterile space, and into a sterile space. To bring a complete set of implants from storage prior to surgery may require several people to carry all the implant caddies.

There is a need for a carrier that provides for sterile storage, that provides for organized screws and other orthopedic implant storage, that provides for easy and safe transportation, and is easily portable.

### SUMMARY

An orthopedic implant carrier system has a carrier with a front cover, a back cover, and a linking member pivotally connected to the front cover and the back cover, such that the front cover and back cover are opposably movable about the linking member, with respect to each other. At least one insert connector is connected to the carrier, with an insert releasably connectable to the at least one insert connector. The insert has a pocket, with a plurality of orthopedic implants organized by implant type, size, and length.

An orthopedic implant carrier system has a plurality of inserts, releasably connected to a closable carrier, with each insert of the plurality of inserts having a plurality of pockets on each insert of the plurality of inserts configured to hold a plurality of containers. The plurality of containers, each having a double layer of enclosing material enclosing an orthopedic implant. Each pocket of the plurality of pockets has a label corresponding to the orthopedic implant type, size, and length of an orthopedic implant held therein.

A method for transporting orthopedic implants includes providing a carrier having a front cover, a back cover, at least one insert connector, and a linking member flexibly connected to the front cover and the back cover such that the front cover and back cover are opposably movable about the linking member. A plurality of inserts are provided, with each insert having a closable pocket. Connecting the plurality of inserts to the carrier using at least one insert connector and a plurality of orthopedic implants are provided, with each implant being enclosed in a container having a double layer of enclosing material, the inner layer and the implant being sterile. The implants are placed into the closable pocket such that the pocket of each of the plurality of inserts has implants of a given type and a given size. The inserts are organized and connected into the carrier such that the inserts are organized by implant type and implant size. The carrier is closed, such that the plurality of inserts are enclosed within the carrier, and the carrier is grasped and moved to a desired location.

A method for removing implants from a carrier including providing a carrier, having a plurality of inserts, each of the plurality of inserts having a plurality of closable pockets. Providing a plurality of implant containers each implant container having an implant within an inner container within an outer container, and the inner container and the implant are sterile. Organizing the plurality of implant containers such that each pocket of the plurality of pockets has implant containers of a particular type, size, and length. Providing an operating room having a non-sterile field and a sterile field. Placing the carrier within the non-sterile field. Opening the carrier and selecting an insert from the plurality of inserts, the insert having a label identifying an implant type, size, and length. Opening the pocket, the pocket containing an implant container from the plurality of implant containers. Removing the implant container. Opening the outer container and having a second person within a sterile field remove the inner container, the second person opening the second container and removing the implant.

An orthopedic implant carrier system includes a carrier having a front cover, a back cover, and a linking member connected to the front cover and the back cover such that the front cover and back cover are opposably movable about the linking member, with respect to each other, the front and back covers are connectably closable.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

FIG. 1 depicts a front view of a carrier, in accordance with an aspect of the present invention;

FIG. 2 depicts a perspective view of the carrier of claim 1, in accordance with an aspect of the present invention;

FIG. 3 depicts the carrier of claim 1 in an open position, in accordance with an aspect of the present invention;

FIG. 4 is a top view of an insert having two pockets of the carrier of FIG. 3;

FIG. 5 is a top view of the insert of the.3 with a flap of the insert open;

FIG. 6 is a top view of the insert of FIG. 5 with a container being inserted into a pocket of the pockets of the insert;

FIG. 7 is a top view the carrier of FIG. 1 opened and having multiple inserts; and

FIG. 8 is a perspective view of an implant being inserted into the container of FIG. 6.

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

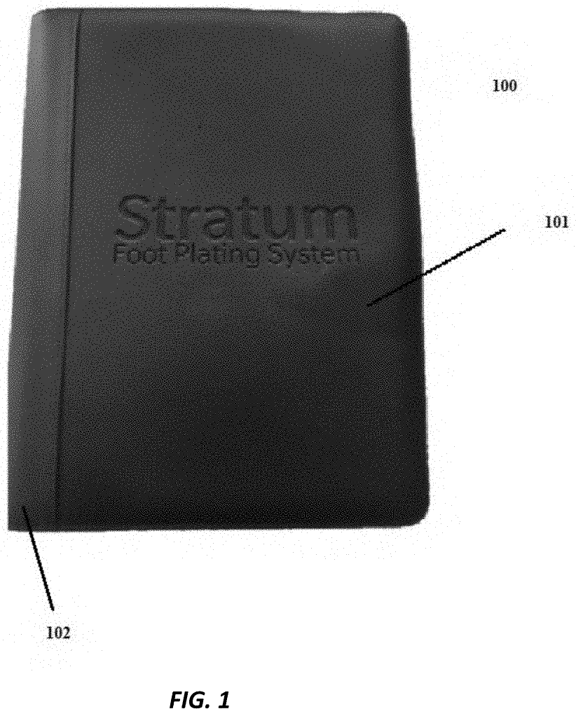

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

The following description references systems, methods, and apparatuses for storing, organizing, and transporting orthopedic surgical implants. However, those possessing an ordinary level of skill in the relevant art will appreciate that other medical objects, surgical tools, and surgical devices are suitable for use with the foregoing systems, methods and apparatuses. Likewise, the various figures, steps, procedures and work-flows are presented only as an example and in no way limit the systems, methods or apparatuses described to performing their respective tasks or outcomes in different time-frames or orders. The teachings of the present invention may be applied to any small medical object.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The various embodiments described herein provide for apparatuses, devices, and methods for storing, organizing, and transporting, orthopedic implants. Particularly, for transportation between non-sterile and sterile environments (e.g., from storage and preparation to a surgical operating room), promote easy transportation, and provide implant organization promoting more efficient preparation in a surgical environment.

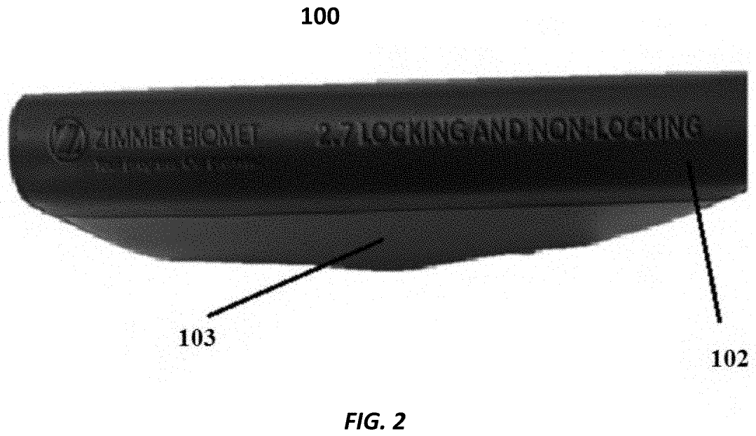

Referring to FIGS. 1 and 2, a carrier 100 is depicted having a front cover 101, a linking member 102, and a back cover 103. Linking member 102 connects to both front cover 101 and back cover 102, forming, for example, a portfolio. Linking member 102 may be, for example, a piece of material or fabric. Linking member 102 may also be, for example, a spine as for a book or binder. Alternate embodiments of carrier 100 may include, for example, a binder or a case. With reference to front cover 101 and back cover 102, front and back may be interchangeable in some embodiments.

Still referring to FIGS. 1 and 2, carrier 100 is depicted in a closed position. Carrier 100 may be treated and may be sterile. To maintain a sterile exterior, carrier 100 may be covered in, for example, a shrink wrap (not shown) or vacuum sealed packaging which may be opened upon entry into a sterile environment, such as a surgical theater.

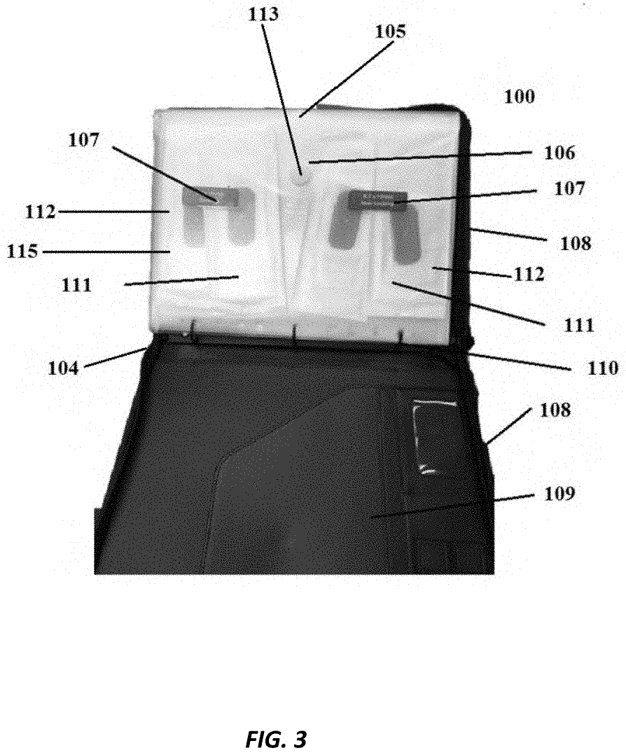

FIG. 3 depicts carrier 100 in an open position. An insert 105 is shown connected to connectors 110. Three connectors are depicted, with connectors 110 affixed to a linking member inner surface 104. In other embodiments, connectors 110 may be connected to a front cover inner surface 109 or a back cover inner surface (not shown). In further embodiments, there may be more than three connectors 110 or as few as one instance of connectors 110. Only one insert 105 is depicted but insert 105 may be one of a plurality of inserts 120, which are stackable and connectable to connectors 110.

Referring to FIGS. 3-6, insert 105 is depicted as having an insert closure 106, a plurality of insert pockets 112 (e.g., two insert pockets), each of which may include a label 107. The number of insert pockets 112 may be specific to a size and type of an implant being contained, but two insert pockets may be most common. There may also be embodiments with a single pocket or there may be several pockets 112 in insert 105. A plurality of implant containers for holding surgical implants are visible within the insert pockets 112 of insert 105. Insert 105 may be made from a clear material (e.g., clear plastic) or have clear pockets 112. Label 107 may be, for example, positioned on an outer face of insert 105, or could form tabs at a top edge or at a side edge of insert 105 or at a combination of positions.

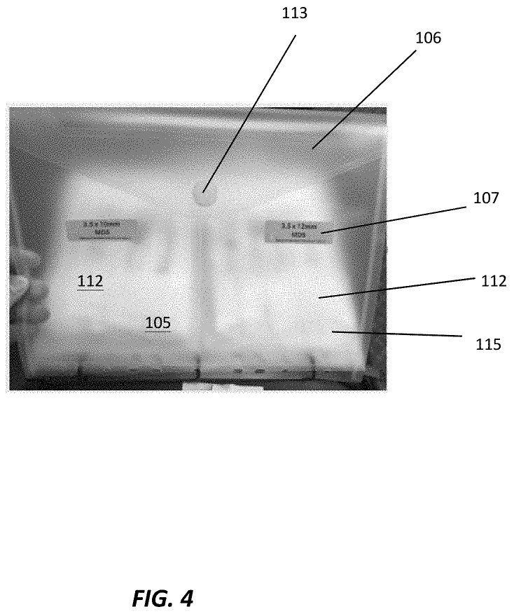

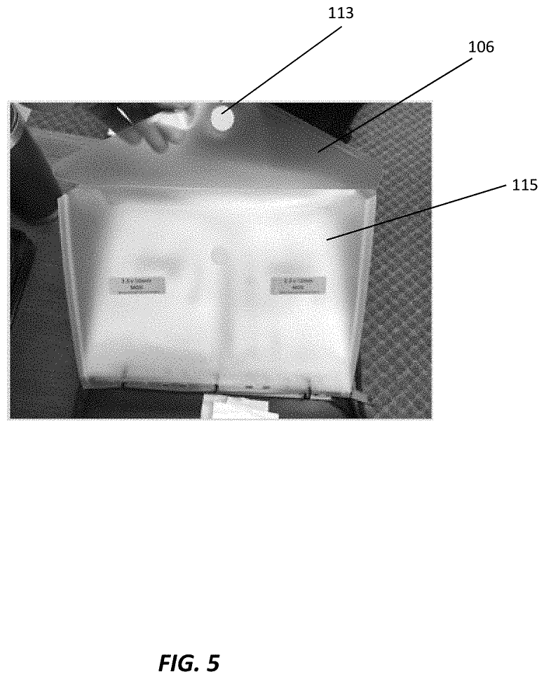

Insert closure 106 may be a flap (e.g., closable portion of material connected to a remainder of insert 105) with a flap connector 113 as depicted in FIGS. 4-5, for example. Insert closure 106 is depicted as a single flap that covers two pockets of insert pockets 112. Insert closure 106 may be a single flap covering a single pocket on insert 105 or a plurality of pockets 112. Flap connector 113 may be, for example, a hook and loop fastener, such as a Velcro® tab, with one end of the Velcro® tab on insert closure 106 and an opposite end on the face of insert 105. Insert closure 106 may also be, for example, a closable opening in insert 105 such that the opening may, for example, be closed via hook and loop (e.g., Velcro®) strips at the opening, a zipper, a press-seal strip, or a slide-seal. In an example, insert 105may include an implant container 111 sealed within an insert pocket of the plurality of insert pockets 112, with the insert pocket being sealed and not having an opening. Thus, it would be necessary to unseal, open, or tear insert 105 to access inserts within insert pockets 112 in this example.

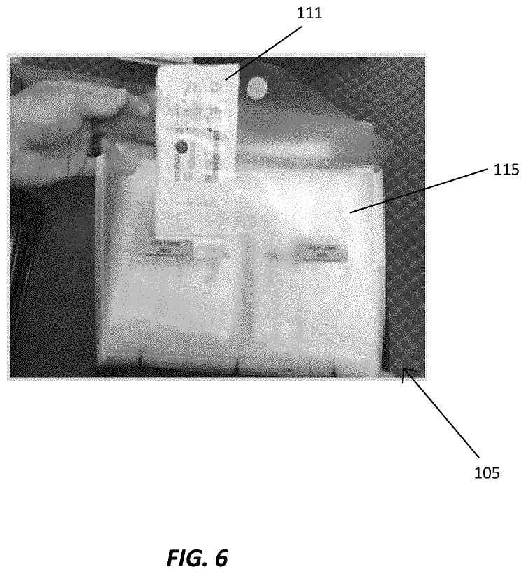

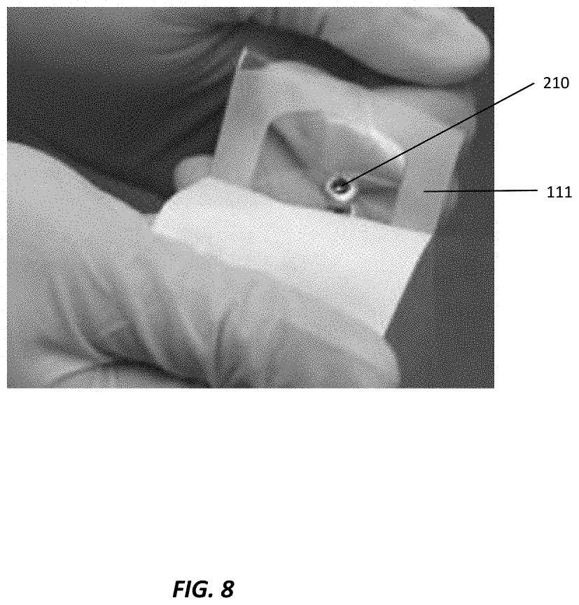

Further referring to FIGS. 3, 6 and 8, implant container 111may be a bag or packet of a flexible material, for example, and may be received in insert 105. Each insert pocket of the plurality of insert pockets 112 may have one or more implant container 111. Any type or style of implant container 111 may be placed into an insert pocket 115 of plurality of insert pockets 112, as depicted in FIG. 6, for example. Such containers (e.g., implant container 111) could be bags, pouches, tubes or boxes, for example. Implant container 111 may be arranged by a size of an implant or implants held within. FIG. 8 depicts an implant 210 being inserted into container 111. Each insert pocket of the plurality of insert pockets 112 may contain, for example, one or more instances of implant container 111 having a single implant size of a single type or style contained within, with label 107 indicating the implant size and type or style. Label 107 may also include an indication of a recommended number of the implant and/or implant container to be held in the insert pocket to which it is attached.

Referring generally to FIGS. 1-3, implants (e.g., implant 210) to be held in implant container 111 and one or more insert pockets 112 may include, for example, a screw, a nail, a rod, a pin, a prosthesis, or a bone plate. With carrier 100 arranged for carrying surgical screws, carrier 100 may be configured (e.g., shaped, dimensioned and arranged) to hold a particular style of screw. Screw styles may include, for example, locking, non-locking, variable angle locking, and cannulated. Other surgical screw types may be included. Screws may be organized, for example, by screws of the same or different diameters. Each insert 105 of a plurality of inserts (not shown) may then be further organized by, for example, length of the screws. Insert pockets 112 may be arranged to allow, for example, two lengths or two styles of screws to be within insert 105. One or more instances of implant container 111 may be arranged to have, for example, all locking screws, with 2.7 mm diameter screws in a first insert pocket of the plurality of insert pockets 112 and 3.5 mm diameter screws in a second insert pocket of a plurality of insert pockets 112 on insert 105. Lengths of such screws to be received in the pockets may range from 12 mm-50 mm, for example, with each insert 105 corresponding to a particular length. A plurality of inserts may be arranged by lengths with each insert 105 arranged in 2 mm increments, relative to each other insert, and containing screw lengths from 12 mm-50 mm. Screws may be organized such that common screw are located in in particular pockets (e.g., pockets 112) and inserts (e.g., insert 105) and such organization may vary according to user preference; however common organization and grouping may be by, for example, style, diameter, and length so that a required screw can be easily found when it is called for in an operating theater. Similarly, carrier 100 may be configured to organize implants other than screw by particular pockets (e.g., pockets 112) and inserts (e.g., insert 105).

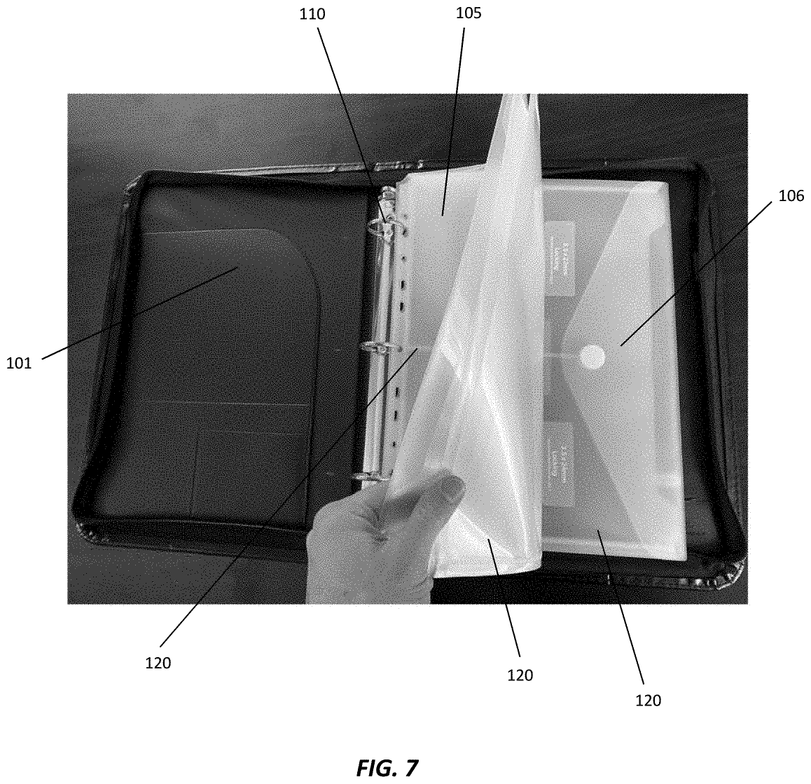

Insert 105 (FIGS. 3-7) may have different configurations, with at least a single insert pocket (e.g., pocket 115) sized to hold multiple implant containers 111 of a particular type of implants. For example, insert 105 with insert pockets 112 configured (e.g., shaped and dimensioned) to hold screws, may have pocket sizes (e.g., a length, width and depth) to accommodate 1-4 types and sizes of screws. Insert 105 may have less insert pockets (e.g., 1-2) if insert pockets 112 are configured (e.g., shaped and dimensioned) to hold, for example, bone plates, versus being configured to hold screws.

Prior to performing an orthopedic surgical procedure, it may not be certain what implant size or type may be needed. Thus, it is often necessary to have many implants of multiple types, styles, diameters, and sizes available in an operating room. Orthopedic surgical procedures may require a large number (e.g., upwards of 400) of implants (e.g., implant 210) to be available for use during a surgical procedure due to the number of scenarios and variables that may arise. Insert 105 may have, for example, two pockets, holding screws of a particular type and length but separated into a particular diameter, for each of the two pockets. Each carrier 100 may hold a plurality of inserts (e.g., insert 105), such that the plurality of inserts may be organized, for example, as screws in 2 mm increments. The embodiment shown in FIGS. 1-3 may, for example, hold approximate 150 screws, resulting in carrier 100 being about 2 inches thick. Three instances of carrier 100 may provide a set of implants for a surgical procedure and make all such screws available to surgeons in an operating room without it being necessary for one to leave the sterile environment to get more. Other embodiments may provide for thicker carriers (e.g., thicker than carrier 100) with more inserts (e.g., insert 105), such that a single carrier 100 may hold a full set of screws or implants.

In one embodiment, carrier 100 may be a case configuration, such as, for example a briefcase.

Still referring to FIG. 3, cover closure 108 may include, for example, a hook and loop fastener (e.g., Velcro®), a zipper, a press-seal strip, or slide-seal, a clasp, or a lock such that front cover 101 and back cover 103 may be connected by cover closure 108 when cover closure 108 is engaged. Cover closure 108 may connect front cover 101 and back cover 103.

Further referring to FIGS. 3-7, insert 105 may have rigid or flexible backing. Insert pockets 112 may be flexible on one side but due to the presence of rigid backing within insert 105, insert pockets 112 may be prevented from flexing or expanding towards back cover 103. Insert 105, front cover 101, and/or back cover 103 may be reinforced to provide additional protection for implants within the carrier 100. The contents of insert 105 may be moved within the flexible material of insert pockets 112 to provide a distribution of the implant containers to utilize the flexibility of insert pockets 112. The presence of rigid backing may provide sturdiness or structure for insert 105 and provide for easier stacking and organization within carrier 100. In the case of insert 105 having a flexible backing, insert pockets 112 may be flexible in all directions. The contents of insert 105 may be moved within the flexible material of insert pockets 112, with the flexibility providing for some content distribution due to stacking. For example, in a stack of inserts (e.g., insert 105) with flexible backing, the contents (e.g. implant container 111) of one insert (e.g., insert 105) may be shifted to utilize the flexibility of insert pockets 112 during the stacking of such inserts. Content shifting for each insert pocket of the plurality of insert pockets 112 may occur as subsequent inserts (e.g., insert 105) are stacked.

The use of a hard front cover 101 and back cover 103 minimizes the need for hard casing, such as tubes or boxes, commonly used for the storage and transportation of individual orthopedic implants. Without hard casing being utilized for implant containers 111, the volume of space required for storing insert containers 111 decreases relative to the use of more rigid materials, such as tubes or boxes. Eliminating such hard casing as a material for implant containers 111 also minimizes waste materials.

Referring to FIGS. 1-8, a method for carrying orthopedic implants includes carrier 100 having front cover 101, back cover 103, linking member 102 flexibly connected to front cover 101 and back cover 103, such that front cover 101 and back cover 103 are opposably movable about linking member 102. Carrier 100 may include a plurality of inserts with each insert being similar or identical to insert 105 and having closable insert pockets 112. Insert closure 106 may be formed as a flap to provide closure to a plurality of insert pockets 112 of an insert (e.g., insert 105). Orthopedic implants may be enclosed in insert containers 111 having, for example, a double layer of enclosing material. Implant containers 111 may be placed into insert pockets 112 such that each one of the plurality of insert pockets 112 of each insert (e.g., insert 105) has, for example, multiple implants of a particular type and size. Implant containers 111 may have different configurations, depending on the implant contained within. Implant container 111 may have a double layer of enclosing material, but the size may vary. For example, implant containers 111 for screws may be approximately the same size for all screw lengths and/or diameters of a given type of screw, but implant containers 111 for bone plates may be larger than implant containers 111 used for screws. Inserts (e.g., insert 105) may be organized by size, style, and type of implant. The interior of implant containers 111 having the double layer configuration may be sterile to maintain sterility of an implant held therein. Implant containers 111 may be, for example, a double sealed pouch, or a double sealed tube, or a double sealed blister pack. Implant containers 11 with a double sealed pouch configuration may be configured to lay flat and would enable more implants to be placed into insert 105 relative to a configuration utilizing a double sealed tube or pouch, or a single layer pouch with a tube or other more rigid construction. Implant containers 111 formed as a double sealed pouch may thus also obviate the need for using internal tubes or boxes to further seal the implant.

Continuing with the method for carrying orthopedic implants, inserts (e.g., insert 105) may be connected to connector 110 in carrier 100. Carrier 100 may be closed such that the plurality of inserts (e.g., insert 105) are enclosed within carrier 100. Cover closure 108 may be engaged. Carrier 100 may then be wrapped in, for example, a blister shell. Carrier 100 may also be vacuum sealed in the blister shell (not shown).

Carrier 100, the exterior blister shell, and the contents may be made from materials that are gamma irradiation stable, allowing for radiation sterilization of carrier 100 and the contents, without having to remove implant containers (e.g., implant container 111) from insert pockets 112. Carrier 100 at this point may be treated in a gamma irradiation process to make carrier 100 and the contents therein sterile. Carrier 100 may then be transported to a desired location, with only the blister shell being exposed to the outside environment.

In another embodiment, implant containers (e.g., implant container 111) may be previously sealed within insert pockets 112. If carrier 100 and the contents are made from materials that are gamma irradiation stable, the contents of carrier 100 and the contents of each insert 105 may be sterilized via gamma sterilization.

A surgical operating room may have a sterile field and a non-sterile field. When carrier 100 arrives in a non-sterile field, the blister shell may be removed, and carrier 100 may be moved to a desired location within a sterile field. With the non-sterile blister shell being disposed of, carrier 100 including its contents may be left sterile.

In still another embodiment, implant containers (e.g., implant container 111) may be gamma irradiated so that the inner container and the implant are sterilized but the outer container may be exposed to a non-sterile environment. In this configuration, carrier 100 may just be a non-sterile container and organizer for implant containers (e.g., implant container 111), while the interior contents of implant container remain sterile.

In an embodiment where implant containers (e.g., implant container 111) are sealed within insert 105, the desired insert may be opened within a non-sterile area of an operating theater and the sterile contents may be taken by a person standing within the sterile field of the operating theater. For example, sterile implant container 111 may be removed, from multiple such implant containers (e.g., implant container 111) for use in the surgical procedure.

In an embodiment where implant container 111 is sterilized such that the outer container remains non-sterile while the interior container and the implant are sterile, carrier 100 may be brought within the non-sterile area of the surgical theater, a desired insert (e.g., insert 105) may be selected. Implant container 111 may be selected and the outer container opened, so that the sterile inner container and sterile implant may be passed to a person within the sterile field for use in the surgical procedure.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthopedic implant carrier system comprising:
a carrier having a front cover, a back cover, and a linking member pivotally connected to the front cover and the back cover, such that the front cover and back cover are opposably movable about the linking member, with respect to each other;
at least one insert connector connected to the carrier;
an insert releasably connectable to the at least one insert connector, the insert having a pocket;
a plurality of orthopedic implants organized by implant type, size, and implant length in the pocket.

2. The orthopedic implant carrier system of claim 1 wherein the carrier is closable.

3. The orthopedic implant carrier system of claim 2 wherein the front cover and the back cover are connectably closable.

4. The orthopedic implant carrier system of claim 1 wherein the linking member is flexibly connected to the front cover and the back cover.

5. The orthopedic implant carrier system of claim 1 wherein the insert has a plurality of closable pockets.

6. The orthopedic implant carrier system of claim 1 wherein the insert has a single pocket divided into two separate parts, and wherein the pocket is closable.

7. The orthopedic implant carrier system of claim 1 wherein each implant is enclosed in a container and wherein the container has a double layer of enclosing material.

8. The orthopedic implant carrier system of claim 1 wherein the container and the implant are sterilizable.

9. An orthopedic implant carrier system comprising:
a plurality of inserts releasably connected to a closable carrier;
a plurality of pockets on each insert of the plurality of inserts configured to hold a plurality of containers;
the plurality of containers, each having a double layer of enclosing material enclosing an orthopedic implant;
wherein each pocket of the plurality of pockets inserts having a label corresponding to the orthopedic implant type, size, and length of an orthopedic implant held therein.

10. The orthopedic implant carrier system of claim 9 wherein each pocket of the plurality of pockets is closable.

11. A method for transporting orthopedic implants comprising:
providing a carrier having a front cover, a back cover, at least one insert connector, and a linking member flexibly connected to the front cover and the back cover such that the front cover and back cover are opposably movable about the linking member;
providing a plurality of inserts, each insert of the plurality of inserts having a pocket;
connecting the plurality of inserts to the carrier using at least one insert connector;
providing a plurality of orthopedic implants;
wherein each implant of the plurality of implants is enclosed in a container having a double layer of enclosing material, an inner layer of the double layer and the implant being sterile;
placing implants into the pocket of each insert of the plurality of inserts such that the pocket of each insert of the plurality of inserts has implants of a given type and a given size;
organizing the inserts and connecting the inserts into the carrier such that the inserts are organized by implant type, size, and length;
closing the carrier, such that the plurality of inserts are enclosed within the carrier;
grasping the carrier and moving the carrier to a desired location.

12. The method of claim 11, where the step of organizing the inserts and connecting the insert into the carrier further comprises sterilizing the carrier.

13. The method of claim 11, where the step of grasping the carrier and moving the carrier to a desired location comprises moving the carrier through a non-sterile location to a sterile location.

14. The method of claim 13, further comprises opening the carrier such that the inserts remain sterile.

15. A method for removing implants from a carrier comprising;
providing a carrier having a plurality of inserts, each insert having a plurality of pockets;
providing a plurality of implant containers, each implant container having an implant within an inner container within an outer container;
wherein the inner container and the implant are sterile;
organizing the plurality of implant containers such that each pocket of the plurality of pockets has implant containers of a particular type, size, and length;
providing an operating room having a non-sterile field and a sterile field;
placing the carrier within the non-sterile field;
opening the carrier;
selecting an insert from the plurality of inserts, the insert having a label identifying an implant type, size, and length;
opening the pocket, the pocket containing an implant container from the plurality of implant containers;
a first person removing the implant container;
opening the outer container;
a second person within a sterile field removing the inner container;
opening the second container and removing the implant.

16. An orthopedic implant carrier system comprising:
a carrier having a front cover, a back cover, and a linking member connected to the front cover and the back cover such that the front cover and back cover are opposably movable about the linking member, with respect to each other;
wherein the front and back covers are connectably closable.

17. The orthopedic implant carrier system of claim 16 further having at least one releasable insert connector.

18. The orthopedic implant carrier system of claim 17 having an insert releasably connectable to the carrier;
wherein the insert has at least two closable insert pockets.

19. The orthopedic implant carrier system of claim 17 wherein a plurality of inserts are organized by implant type, size, and length.
